(19) United States
(12) Patent Application Publication
(10) Pub. No.: US 2019/0214123 A1
(43) Pub. Date: Jul. 11, 2019

(54) A method and a system for storing drugs in distribution packages, and a storage for drug distribution packages

(71) Applicant: NewIcon Oy, Kuopio (FI)

(72) Inventor: Ossi PARVIAINEN, Kuopio (FI)

(21) Appl. No.: 16/328,736

(22) PCT Filed: Sep. 5, 2016
(86) PCT No.: PCT/FI2016/050614

§ 371 Date: Feb. 27, 2019

(51) Int. Cl. G16H 20/13; G06Q 10/08; A47F 3/02; A47F 3/00; A47B 88/988

(52) U.S. Cl. CPC G16H 20/13; G06Q 10/087; A47B 81/00; A47F 3/002; A47B 88/988; A47F 3/02

(57) ABSTRACT

A system for storing drugs in distribution packages of a storage for drug distribution packages includes a storage for drug distribution packages, including storage locations, where the distribution packages are placed; a data processing device with a memory for storing the number of distribution packages and/or drugs in the storage for drug distribution packages, the expiry date of the drugs, and at least one item of drug information and the personal data of a user of the storage for drug distribution packages; a stock data system stored in the memory of the data processing device configured to control the functions of the storage for drug distribution packages and to record stock data entries in the storage for drug distribution packages and to administer user rights of the stock data system; at least one detecting sensor placed in storage locations coupled to the data processing device.

### FIELD OF THE INVENTION

The invention relates to a method and a system for storing drugs in distribution packages, and a storage for drug distribution packages.

### BACKGROUND OF THE INVENTION

In many countries, it is common drug distribution practice in hospitals, health clinics and nursing homes that the drugs to be administered to patients are unpacked from the drug packages into distribution packages to be stored in a storage for drug distribution packages, from which they are administered to patients according to their treatment regimen. This is done because it is often thought that it is easier to take loose drugs from a distribution package than from the original drug package of the pharmaceutical manufacturer where e.g. pills are often in bubble packs, whereby the drugs have to be detached one by one before they are administered to patients.

A disadvantage in such a method is that after the unpacking and transfer of the drugs into distribution packages, the expiry date of the drugs is normally no longer known, because this same drug may have been added to the same distribution package from several different drug packages, each of which may have a different expiry date for the drugs. Yet another disadvantage in such a method for storing and distributing drugs is that it is possible to abuse the drugs, because the taking of drugs placed in the distribution packages is not prevented in any way, and the taking of drugs from the distribution packages is often not monitored in any way either.

### BRIEF SUMMARY OF THE INVENTION

It is an aim of the invention to provide a novel system for storing drugs in distribution packages, whereby information about the expiry date of the drugs stored in the distribution packages is maintained even though the drugs are transferred from the original drug package of the manufacturer to a distribution package, and whereby the abuse of drugs placed in the distribution packages can be prevented better than before. Furthermore, it is an aim of the invention to introduce a method for storing drugs in distribution packages, and a storage for drug distribution packages, which can be implemented in the system according to the invention.

The aim of the invention is attained by a system in which the adding of drugs into a distribution package requires user identification (that is, logging in the data system of the storage for drug distribution packages), and by which the date information and/or the production batch number of the drugs in the drug package is entered in real time and automatically or manually so that the data of the drugs, the expiry date and/or the production batch number are entered in the storage data system of the storage of drug distribution packages when drugs are added into distribution packages. Furthermore, the drug distribution package belonging to the system is structurally implemented in such a way that the addition or removal of drugs to or from distribution packages requires that those distribution packages which are involved in removing or adding drugs, are removed from the drug distribution storage or are at least moved, so that this will induce a stock change entry in the stock data system of the storage of drug packages, by a detecting sensor in the storage of drug packages. Thus, the stock data system of the storage for drug distribution packages always has up-to-date information on the expiry date and/or the production batch number of the drugs in the distribution packages, as well as on the location of the different drugs in the storage for drug distribution packages. Thus, it is not possible to add or to remove drugs to or from the distribution packages without entering a stock change in the stock data system of the storage for drug distribution packages. To put it more precisely, the system according to the invention is characterized in what will be presented in the independent claim 1. To put it more precisely, the method according to the invention is characterized in what will be presented in the independent claim 8, and the system according to the invention is characterized in what will be presented in the independent claim 12. Dependent claims 2 to 6 present advantageous embodiments of the system according to the invention, dependent claims 9 to 11 present advantageous embodiments of the method according to the invention, and dependent claims 12 to 15 present advantageous embodiments of the storage for drug distribution packages according to the invention.

The system, the method, and the storage for drug distribution packages according to the invention have the advantage that they make sure that the drugs stored in the storage for drug distribution packages are never out-of-date, and on the other hand, the drugs do not need to be disposed of too early because of older drugs (having an earlier expiry date or old production batch number) mixed with newer drugs in the same distribution package. Furthermore, the system, the method, and the storage for drug distribution packages according to the invention improve drug safety by preventing abuse of drugs in the distribution packages in the storage for drug distribution packages, without leaving an entry in the stock data system of the storage for drug distribution packages, about the time when and by whom drugs have been taken from the storage of drug distribution packages.

### DESCRIPTION OF THE DRAWINGS

In the following, some advantageous embodiments of the invention will be described in more detail with reference to the appended drawings, in which

FIG. 1 shows a storage for drug distribution packages according to the invention, in a slanted view from above;

FIG. 2 shows a drawer for a storage for drug distribution packages according to FIG. 1, containing distribution packages to be stored in it, in a slanted view from above;

FIG. 3 shows a drawer for the storage for drug distribution packages according to FIG. 1, seen from above, all the distribution packages being placed in their storage locations in the drawer;

FIG. 4 shows a drawer for the storage for drug distribution packages according to FIG. 1, seen from above, all except one of the distribution packages being placed in their storage locations in the drawer;

FIG. 5 shows one distribution package for the storage for drug distribution packages according to FIG. 1, with a lid, and a detecting sensor in a storage location, the distribution package being apart from the detecting sensor;

FIG. 6 shows the detecting sensor in the storage location for the distribution package according to FIG. 3, the distribution package being placed in its storage location;

FIG. 7 shows, in a slanted view from the front, another storage for drug distribution packages, similar to the storage for drug distribution packages shown in FIGS. 1 to 6, where the distribution packages of the storage for drug distribution packages according to FIGS. 1 to 6 are placed in a horizontal position, on top of and next to each other;

FIG. 8 shows a third version of a storage for drug distribution packages according to the invention, seen in a slanted view from above;

FIG. 9 shows a fourth version of a storage for drug distribution packages according to the invention, seen in a slanted view from above; and

FIG. 10 shows a fifth version of a storage for drug distribution packages according to the invention, seen in a slanted view from the front.

### A DETAILED DESCRIPTION OF PREFERRED EMBODIMENTS OF THE INVENTION

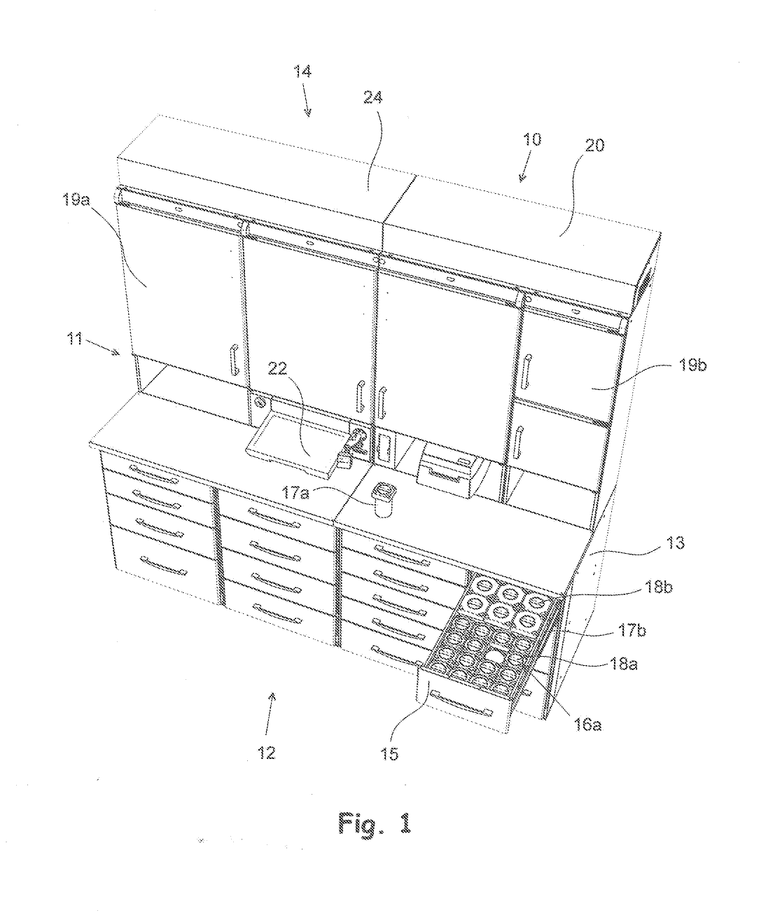

FIG. 1 shows a storage 10 for drug distribution packages according to the invention. It comprises a frame 11 whose lower part comprises a chest 12 of drawers comprising several drawers 15 placed on top of and next to each other, and lockable by electric locks. The drawers 15 have storage locations 16a and 16b for distribution packages 17a and 17b to be placed in them. On top of the chest of drawers 12, the frame 11 also comprises a counter top 13 and a cabinet 14 on top of it. The cabinet 14 comprises shelves with storage locations for distribution packages to be placed next to and on top of each other in the horizontal plane. The counter top 13 comprises a table board which is used as a desktop when adding and removing drugs into and from distribution packages 17a and 17b. The storage 10 for drug distribution packages shown in FIG. 1 also comprises a data processing device 20 and a stock data system for controlling the operation of the storage for drug distribution packages as well as the adding and removing of drugs or other medical products to and from distribution packages by software.

The frame 11 is made of e.g. sheet metal, plastic, chipboard, composite, plywood, solid wood, or another suitable material, or a suitable combination of these. In an embodiment of a large size, the frame 11 may also comprise a skeleton formed of beams, to which its outer walls are fastened, or separate connecting or fastening elements, by which the outer walls are joined to each other. As a mechanical structure, the skeleton 11 is implemented in such a way that the walls belonging to it surround the chest 12 of drawers and the cabinet 14 so that the distribution packages 17a and/or 17b stored in the chest 12 of drawers can only be accessed by pulling out the drawers 15 out of the chest 12 of drawers, and the distribution packages of the cabinet 14 can only be accessed by opening the doors of the cabinet. Furthermore, the drawers of the chest 12 of drawers and the cabinet doors 19a of the cabinet 14 are equipped with electric locks, by which the drawers 15 of the chest 12 of drawers and the cabinet doors 19a of the cabinet 14 can be locked by programming the data processing device 20, to allow access to the distribution packages 17a and 17b placed in them by authorization only.

The storage locations 16 in the drawers 15 of the chest 12 of drawers are determined according to the size of the distribution packages 17a and 17b used, as well as the length and the width of the rectangular lids 18a and 18b belonging to them. The length and the width of the box-like space formed inside the drawers 15 is dimensioned so that a determined equal number of distribution packages 17a and/or 17b can be placed adjacent to each other in the width and the length direction in each drawer 15. In the drawer 15, the lids 18a and/or 18b of the distribution packages 17a and 17b abut each other in the length and the width directions so that the lids 18a and/or 18b cannot be rotated or the distribution packages 17a and/or 17b cannot be moved in the length or width direction of the drawer 15 when all the distribution packages 17a and/or 17b fitting in a single drawer 15 are placed in the drawer 15. Also, the diameter of the receptacle parts of the distribution packages 17a and 17b and the width of the drawers 15 are selected in such a way that one drawer 15 accommodates a given number of distribution packages (in this case, 4 smaller distribution packages 17a or 3 larger distribution packages 17b) next to each other in so that no empty space is left between the side walls of the drawer but the distribution packages 17a and 17b can be easily fitted in their own storage locations in the drawer, to be manually movable in the vertical direction of the drawer 15. Also, the length of the drawer 15 is determined by a corresponding principle. As shown in FIG. 1, 4 smaller distribution packages 17a and 2 larger distribution packages 17b are fitted in the drawer 15, in its longitudinal direction. Thus, the drawer 15 of FIG. 1 comprises storage locations for 16 smaller distribution packages 17a and for 6 larger distribution packages 17b. The depth of the frame 11 at the chest 12 of drawers is dimensioned so that the drawers 15 fit to be pushed all the way into the chest 12 of drawers.

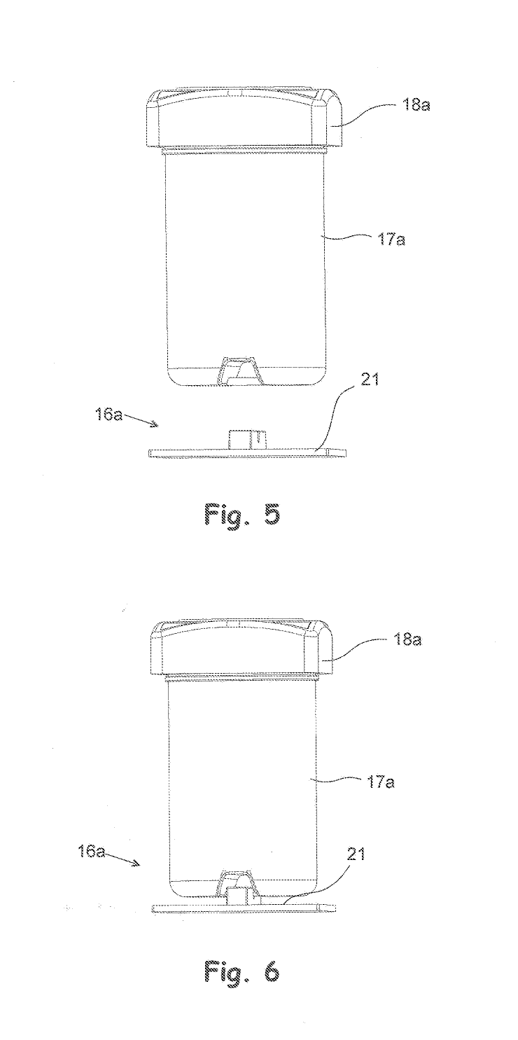

In this embodiment, the distribution packages 17a and 17b to be stored in the chest 12 of drawers are receptacles with a cylindrical cross section, having a lid with a square shape as shown in FIGS. 1 to 6. The receptacle part 23a or 23b, having in this case a cylindrical cross section and being closed with a lid 18a or 18b, may be made of a transparent material (e.g. suitable plastic), as shown e.g. in FIGS. 5 and 6. Inside the lid 18a (and 18b), a cylindrical joining part is provided, having an inside thread matching with a thread formed on the top rim of the receptacle part 23a or 23b (on the outer surface of the side wall of the receptacle part), whereby the lid 18a is fastened to the receptacle part, in this embodiment, by unscrewing the lid 18a or 18b with respect to the receptacle part 23a or 23b. For example, the height of the lid 18a for a distribution package 17a is about 25 mm in the height direction of the distribution package, so that when the distribution package is fitted in the drawer 15, it has to be pulled upwards at least this distance out of the drawer 15 before the lid 18a can be unscrewed so that the lid 18a can be removed from the receptacle part 23 for opening the distribution package 17a.

The cabinet 14 comprises a rack having a rear wall and side walls, as well as vertical intermediate walls and horizontal shelves between these, storage locations being formed between them for distribution packages to be placed in the horizontal direction. The cabinet doors 19a closing the storage locations of the cabinet 14 at the front are equipped with electric locks for controlling access to the distribution packages stored on the shelves of the cabinet. The distances between the side walls and the intermediate walls of the cabinet 14 are selected so that a given number of distribution packages, to be placed next to each other in the horizontal position, fit between each two adjacent vertical surfaces so that hardly any empty space is left between the distribution packages and these surfaces, but the distribution packages can still be fitted in the storage locations formed between the walls and the shelves, to be easily movable manually. In a corresponding way, the horizontal lower wall, the upper wall and the other horizontal shelves between these in the cabinet 14 are placed in such a way that a given number of distribution packages fit between two opposite horizontal surfaces so that hardly any empty space is left between the distribution packages and these surfaces but the distribution packages can still be fitted in the storage locations formed between the walls and the shelves, to be easily movable manually. The cabinet 14 may also comprise one or more non-lockable or lockable cabinet doors 19b for shelves for possibly storing various auxiliary devices and supplies which are needed for the distribution of drugs but whose use does not need to be controlled as such. The depth of the cabinet 14 is normally dimensioned so that the distribution packages, possibly of different sizes, to be placed in it are well accommodated in the cabinet 14 when the cabinet doors are closed.

In the embodiment of FIG. 1, there are vacant compartments 21 which are open on the side of the counter top 13, underneath the cabinet 14 and flush with the counter top 13. As shown in the figure, the touch screen of the data processing device 20 for controlling the storage 10 of drug distribution packages is placed in one of such compartments. The data processing device 20 comprises a central processing unit with a memory (e.g. SSD mass memory and main memory) for storing software controlling the storage 10 for drug distribution packages, and a touch screen 22 by which a user can control the functions of the storage 10 for drug distribution packages. The touch screen 22 is, in the way shown in FIG. 1, configured to extend from the compartment onto the counter top. In this case, the data processing device and other necessary control electronics are placed in an auxiliary housing 24 on top of the cabinet 14. In this case, a stock data system is installed in the memory of the data processing device 20. It controls and administers the storage and distribution of drugs in the storage 10 for drug distribution packages, and e.g. the locking and unlocking of the drawers 15 in the chest 12 of drawers, according to the drugs being taken or added by the person using the storage 10 for drug distribution packages. The stock data system also controls the use, that is, keeps a record of authorized users and keeps a record of entries (removals and additions of drugs in the storage for drug distribution packages) by users.

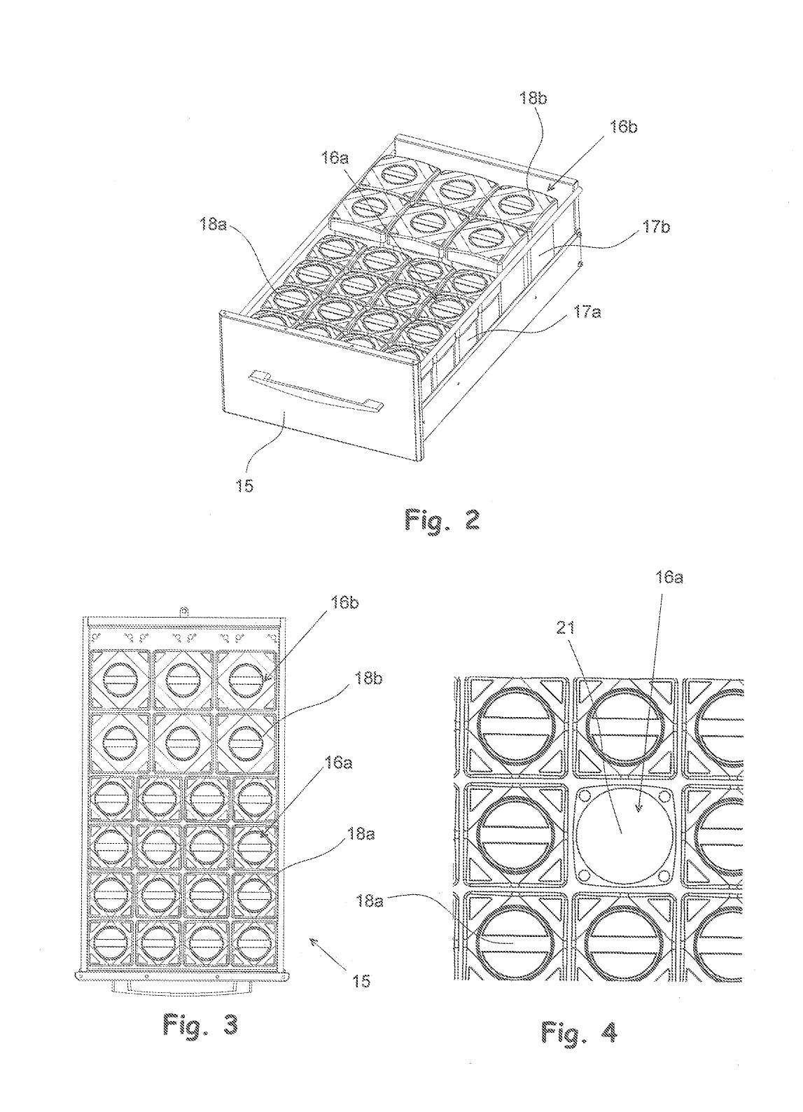

FIGS. 2 and 3 show one drawer 15 in the storage 10 for drug distribution packages according to FIG. 1 in more detail. In this drawer 15, the whole content (all the storage locations 16a) is filled with smaller distribution packages 17a inside, as shown in the figure. As shown in FIGS. 2 and 3, the drawer accommodates 32 such distribution packages 17a. FIG. 3 also shows how the lids 18a of the distribution packages 17a prevent them from being unscrewed when the distribution packages 17a are placed in the storage locations intended for them within the drawer 15. Consequently, in this embodiment, the stop device preventing the addition of drugs into a distribution package 17a or 17b or the removal of drugs from a distribution package 17a or 17b is formed by the lid 18a in the case of the distribution package 17a and the lid 18b in the case of the distribution package 17b.

FIG. 4 shows a situation in which one distribution package 17a has been removed from the drawer 15 shown in FIGS. 2 and 3. Thus, in the drawers 15 according to this embodiment, the storage locations 16a are formed by the spaces determined according to the lids 18a of the distribution packages 17a within the drawer 15. A detecting sensor 21 is placed for each such storage location 16a on the bottom of the drawer 15, for detecting whether the distribution package 17a is placed in the storage location 16a of the drawer 15. In this embodiment, the detecting sensor 21 is an NFC reader whose state is changed and which reads an NFC identifier on the bottom of the distribution package 17a when the distribution package 17a is placed in a storage location 16a of the drawer 15, or when the distribution package is removed from the storage location 16a. All the storage locations 16a in the drawer 15 are equal, so that the dispensing package 17a can be placed in any storage location 16a in the drawer 15.

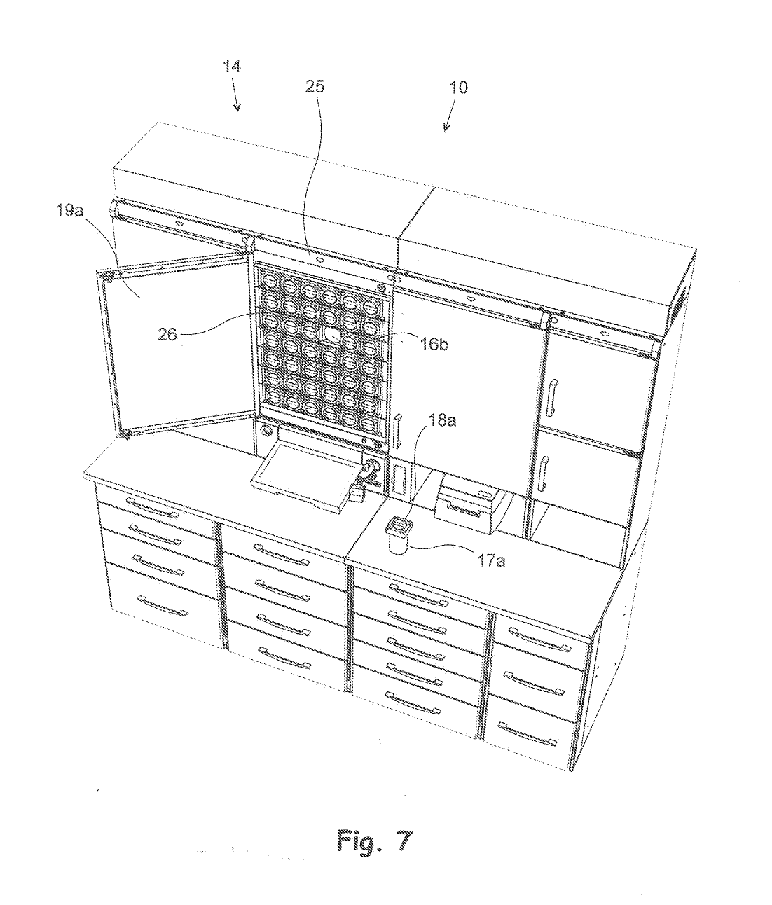

As shown in FIGS. 5 and 6, a detecting sensor 21 is placed on the bottom of the drawer 15 or (particularly in the vertically installed model shown in FIG. 7) on a side of the drawer 15, for each storage location, to be left under the bottom or a side wall of the distribution package 17a. In this case, the detecting sensor 21 is an NFC reader. Therefore, an NFC identifier is provided on the bottom or on a side wall of each distribution package, to be read by an NFC reader used as a detection sensor 21 on the bottom of the drawer 15, when the distribution package 17a or 17b is inserted in or removed from a storage location 16a. The detecting sensor 21 is coupled to the data processing device 20 of the storage for drug distribution packages. Thus, if a distribution package 17a is removed from or inserted in a storage location 16a in the drawer 15, the stock data system is informed about the stock entry in said drawer 15. On the basis of this data, the stock data system concludes that the person who logged in the stock data system and opened said drawer 15 has removed said drug distribution package 17a from the drawer 15 opened at that time. Thanks to the NFC identifiers, the stock data system always identifies each distribution package, and the stock data system always contains up-to-date information on the storage locations 16a of the distribution packages 17a containing each drug, even if the user returned the distribution package 17a to a location different from the location where it was originally taken from.

The stock data system installed in the data processing device 20 may be programmed to operate, for example, in such a way that it always assumes that drugs have been removed from the distribution package 17a or 17b removed, unless the user makes an entry in the stock data system on adding, including the data of the drug package from which drugs were added into the drug distribution package 17a or 17b, as well as the quantity of drugs (e.g. number of pills or tablets). If the added drug quantity and the drug quantity in said drug package do not match, it is concluded that the whole content of said drug package was not added into said drug package 17a or 17b from the other drug package, the system reports a loss (use of drugs off the record), and sends a notice to e.g. a given person responsible for said storage 10 of drug distribution packages, according to the settings. Thus, in this embodiment, the content of a whole package always has to be added to the distribution package 17a or 17b. In this way, the stock data system always contains information on the quantity of drugs in stock, because also when drugs are taken out of the distribution package 17a or 17b, the user has to enter the quantity of drugs taken, which data can be e.g. compared by the system with the dosage according to the regimen of said patients, if the stock data system of the storage 10 for drug distribution packages is connected to a patient data system. Based on this, the stock data system could also determine a time when said drug should be added into said distribution package 17a or 17b, and see that older drugs are used first or removed from said drug distribution package 17a or 17b so that the drugs in this distribution package 17a or 17b will not be mixed with drugs possibly expiring soon, but these drugs will be either used first or disposed of from said distribution package 17a or 17b before newer drugs with a later expiry date are added into it.

The drugs to be stored in the distribution packages 17a or 17b may be pills, tablets, ampoules, or vials for syringes. They may also be small drug packages or also small instruments to be used in medical treatment, for example needles for drug syringes, or other medical equipment fitting in distribution packages. In principle, the only restrictions are the size of the drugs, drug packages or medical equipment to be stored, and the shelf life in the case of pharmaceutical products (e.g. if cold storage or sterile space is required).

For using the storage 10 for drug distributing packages shown in FIG. 1, the user logs in the stock data system of the storage 10 for drug distribution packages. This is done, for example, by entering the user's own user identification and password on the touch screen 22 of the data processing device 20, or the data processing device 20 is connected to a card reader where the authorized user places an electronic ID card and e.g. accepts logging in by touching a key showing this function on the touch screen.

After the login, the touch screen 22 enters a mode in which selection keys are displayed for the user to select which drug or which medical equipment is to be taken from or added into the storage 10 for drug distribution packages. For adding or removing, the menu may include separate keys for first selecting whether drugs are to be removed or added.

If drugs are to be removed, the above mentioned selections are followed by entering the quantity of drugs to be removed from the storage 10 of drug distribution packages. This is followed by unlocking of the drawer 15 or cabinet door 19a where said distribution package 17a or 17b is placed. The location of the unlocked drawer 15 or cabinet door 19a may be displayed on the touch screen. Alternatively or in addition, the unlocked drawer 15 or cabinet door 19a may be equipped with an indicator (e.g. LED) to indicate the drawer 15 where said distribution package 17a or 17b is stored, or the cabinet door 19a behind which said distribution package is stored on a shelf. The touch screen may also display an image of the drawer 15 or rack 19a and point out the storage location 16a of said distribution package 17a or 17b therein. In addition, the stock data system may be programmed to operate in such a way that if the user, in spite of these instructions, picks up a distribution package 17a or 17b from an incorrect location, the stock data system gives a sound alarm (e.g. a beep) and displays the location of the distribution package 17a or 17b which the user tried to pick up, with a red colour on the touch screen 22, and also displays more clearly (e.g. with green colour) the location where the distribution package 17a or 17b should have been picked up. After taking out the desired quantity of drugs, the stock data system requests that the distribution package 17a or 17b be returned to its original location. If, however, the user wishes to place the distribution package 17a or 17b in another vacant storage location 16a, the user may request the stock data system to display a map of vacant storage locations 16a and then select a desired vacant storage location 16, or simply just place the distribution package 17a or 17b in a vacant storage location 16a, wherein the stock data system will update the entry automatically.

For adding drugs, in turn, the stock data system requests for entering data of the drug (or other product) to be added. This can be done manually by entering the data by means of the touch screen, by reading a bar code or a remote readable identifier (e.g. RFID or NFC identifier) in the drug package. The data of the drug to be added into the distribution package 17a or 17b also includes the expiry date of the drug. The stock data system may thus be programmed to operate in such a way that it informs the user of the expiry date of the corresponding drugs in the distribution package 17a or 17b. If the expiry date of these drugs is coming soon (within a predetermined time), the stock data system may ask the user first to take out said drugs from this distribution package so that the soon expiring drugs are not mixed with newer drugs whose expiry date and/or production batch number may be considerably later than that for the drugs present in the distribution package 17a or 17b at the time. The stock data system may thus, for example, suggest that these drugs be placed in another vacant distribution package 17a or 17b, from which it will guide the next taker of these drugs to pick up these drugs first, if they would be used before the expiry date of said drugs. If, on the other hand, the person logged in the system is going to take some of these drugs out of said distribution package 17a or 17b, the stock data system may be programmed to indicate that these drugs have to be removed from said distribution package 17a or 17b first, before newer drugs of the same kind, having a later expiry date and/or production batch number, are added into it. In this way, the stock data system makes sure that the drugs to be stored in the storage 10 for drug distribution packages would be used in such a way that newer drugs to be added in the distribution packages 17a and 17b are not mixed with older drugs, whereby their last expiry date will be determined according to the oldest drug in said distribution package 17a or 17b. However, mixing can be allowed if there is no significant difference (greater than a predetermined interval) between the expiry dates of the drugs to be added and the drugs present in the distribution package 17a or 17b at the time.

After the addition or removal of drugs (or other products as mentioned), the lid of the distribution package 17a or 17b is closed and the distribution package 17a or 17b is returned to the storage location 16a intended for it. After this, the drawer 15 or cabinet door 19a is closed and the user logs out of the stock data system. At this stage, the stock data system controls the electric lock of the drawer 15 or cabinet door 19a to be locked, whereby the distribution packages 17a or 17b will remain in a locked space until the same or another user logs in the stock data system and performs a desired function either to remove drugs (or other products) or to add them in the storage 10 for drug distribution packages.

The system, the method, and the storage for drug distribution packages according to the invention can be implemented, in many respects, in a way different from the example embodiment presented above. For example, the way of implementing the storage locations in the storage for drug distribution packages may vary. In an embodiment, the storage for drug distribution packages may comprise a frame comprising only drawers similar to those in the chest of drawers in the storage for drug distribution packages shown in FIG. 1, or on the other hand, a frame similar to the cabinet, comprising only storage locations formed on shelves to be closed with cabinet doors. The distribution packages themselves may be different in shape (having e.g. a rectangular, square, oval or another polygonal cross-section), and the lid belonging to them may be configured to be opened and closed in a different way (e.g. by pulling to the side or by turning open).

FIG. 7 shows another embodiment of the storage 10 for drug distribution packages according to the invention, in which the distribution packages 17a of the storage 10 for drug distribution packages as shown in FIG. 1 can be stored in a horizontal position. For this purpose, the storage 10 for drug distribution packages of FIG. 7 comprises a compartment 25 placed in the cabinet 14 of the storage 10 for drug distribution packages similar to FIG. 1, equipped with shelves 26 and storage locations 16b on them, in which the distribution packages 17a can be placed next to each other so that the threaded lid 18a of the distribution packages 17a can only be unscrewed by pulling out the distribution package 17a over at least the thickness of the lid from the shelf 26 of the cabinet 25. The embodiment of FIG. 7 may be otherwise similar to the embodiment of FIG. 1, with respect to its principle of operation. The cabinet 25 may also constitute the storage for drug distribution packages without the chest 12 of drawers, and comprise one or more racks of shelves to be closed by a door 19a.

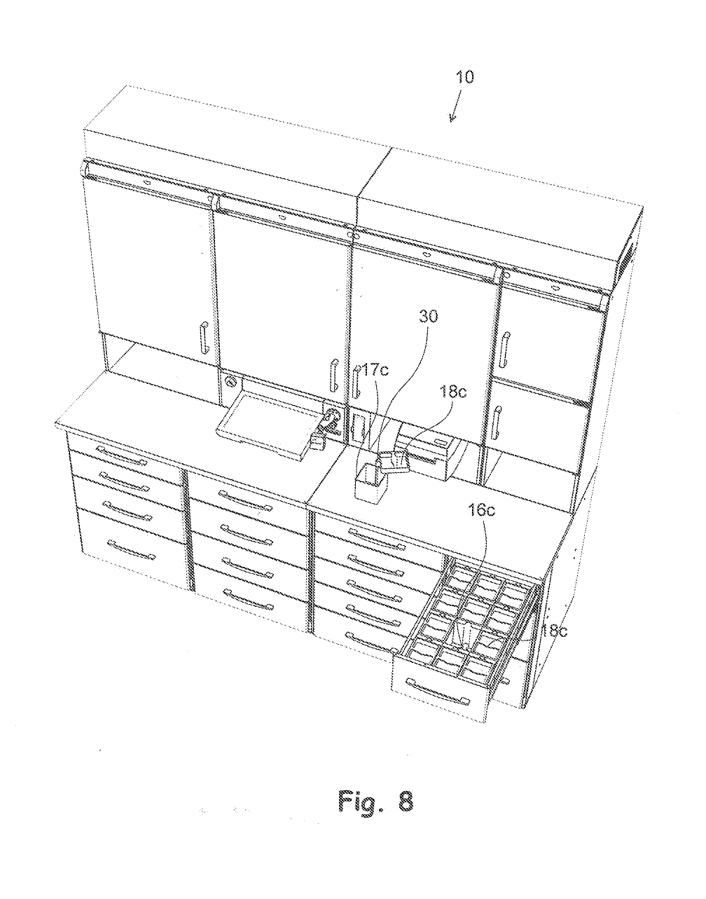

FIG. 8 shows another embodiment of the storage 10 for drug distribution packages according to the invention, in which the lids 18c of distribution packages 17c are unscrewed with respect to a rotating axis 30 transverse to a plane parallel with the lid 18c formed on one side of the distribution package 17c. In this case, the rotation axis 30 is a pin with a circular cross-section, fastened to the outer surface of the side wall of the distribution package and fitted to a bearing retainer connected to the rim of the lid 18c in such a way that the lid 18c can be rotated with respect to the rotation axis 30 in the plane formed by the lid 18c. Thus, the lid 18c can be unscrewed completely off the top of the distribution package 17c so that drugs can be taken out of or added into the distribution package 17c. When closing the distribution package 17c, the lid 18c is screwed back onto the distribution package 17c so that it covers the distribution package 17c whose top it otherwise open, and prevents the removal of drugs from the distribution package 17c. As shown in FIG. 8, twisting the lid 18c is not possible when the distribution package 17c is placed in its storage location in the drawer 15, because the adjacent distribution packages 17c prevent the unscrewing of the lid 18c. Thus, in this embodiment of the system and the storage for drug distribution packages according to the invention, too, the lid 18c constitutes a stop device preventing the removal of drugs from the distribution package 17c and the addition of drugs into the distribution package 17c when it is fitted in its storage location 16c. The storage 10 for drug distribution packages shown in FIG. 8 may also be similar to the embodiment of e.g. FIGS. 1 to 6 in other respects.

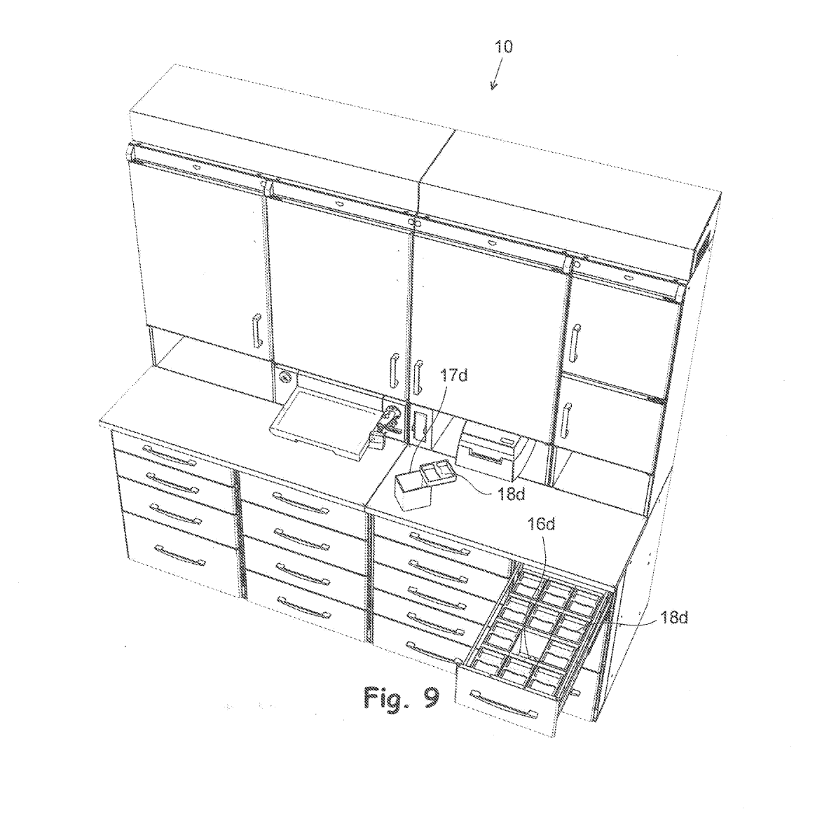

FIG. 9 shows a fourth embodiment of the storage for drug distribution packages according to the invention. In this case, the distribution packages 17d are equipped with a lid 18d that is slidable to the side of the distribution package when opened. The storage locations 16d are placed in a drawer 15 in such a way that the distribution package 17d has to be pulled over at least a given distance (also in this case, approximately the thickness of the lid 18d) out of the drawer 15, for opening its lid 18d. This movement is so large that a detecting sensor in the drawer 15 will detect that said distribution package 17d is being moved and concludes that drugs are being removed from or added into the distribution package 17d. Consequently, in this embodiment of the system and the storage for drug distribution packages according to the invention, too, the lid 18d constitutes a stop device preventing the removal of drugs from the distribution package 17d and the addition of drugs into the distribution package 17d when it is fitted in its storage location 16d. In other respects, this embodiment may also be similar to the embodiment of e.g. FIGS. 1 to 6. In this case, the distribution packages 17d equipped with a slidable lid may also be fitted in storage locations behind cabinet doors 19a in a cabinet 14, because also in them, it is not possible to slide out the lid 18d of the distribution package 17d without pulling the distribution package 17d outwards.

Different ways of opening the lid may require different stop devices for preventing the opening of the lid when the distribution package is placed in the storage location. Also, the method of identifying the distribution packages may be implemented in a way different from the NFC identifiers and NFC readers. In a simpler embodiment, e.g. mechanical micro switches or inductive or optical sensors could be used for picking out distribution packages. However, this would increase the risk of mixing the data of the distribution package if the distribution package is not returned to the same storage location where it was before adding or removing the drugs (or other products) to be stored in it, because such detecting sensors are not capable of identifying if said distribution package is exact the one that was taken out of another storage location earlier. However, if distribution packages are handled one by one, even this kind of an arrangement is relatively reliable, even more so if taking other distribution packages out of the storage for distribution packages is prevented, e.g. by locking devices between the distribution packages and the storage locations.

In an embodiment of the storage for drug distribution packages according to the invention, the distribution packages are open on one side or on top. In this case, the removal of drugs from or adding into the distribution package may be prevented by a stop device which makes it impossible to use a hand, fingers, or instruments suitable for picking up or adding drugs, for adding or removing drugs.

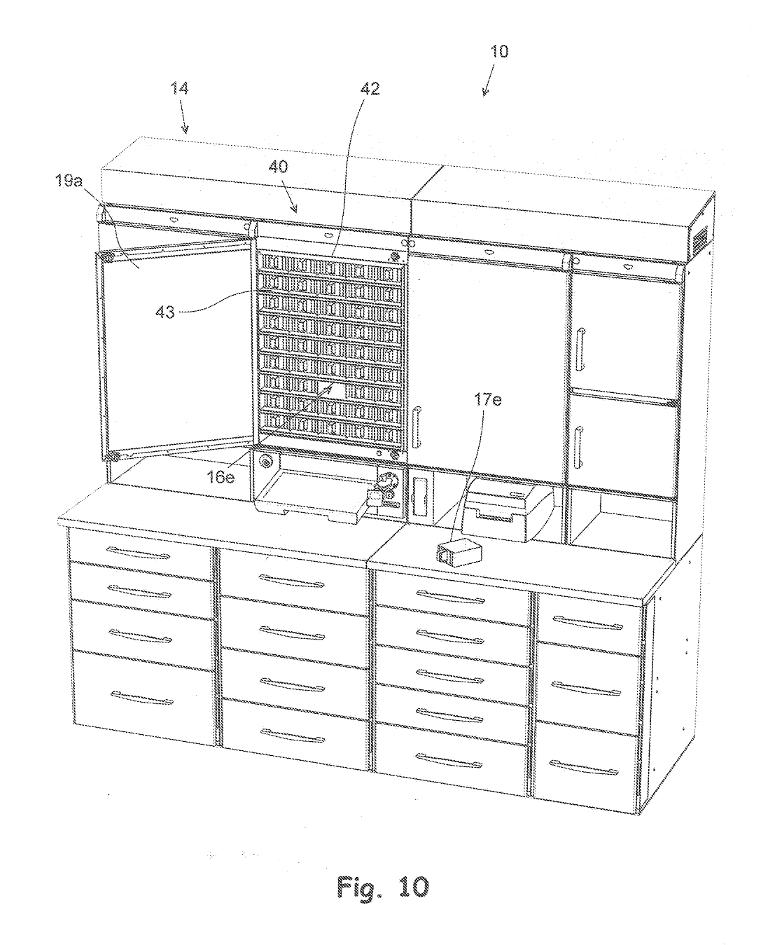

In a fifth embodiment of the storage 10 for drug dispensing packages according to the invention, shown in FIG. 10, a compartment 40 similar to that in FIG. 7 and placed in a cabinet 14 of the storage 10 for drug dispensing packages shown in FIG. 1, may be provided, equipped with a rack 42 comprising storage locations 16e on shelves 43 on top of each other, and distribution packages 17e being fitted in the storage locations 16e via their open front walls. Detecting sensors are placed at the storage locations 16e (one for each storage location 16e) on the rear wall or on the top surface of the shelves 43, for detecting even a relatively small movement of a distribution package 17e from the storage location 16e outwards or inwards. Thus, drugs cannot be added to or removed from the distribution packages 17e without informing the stock data system about a change in the position of the distributing package 17e in said location (which may thus be interpreted as a stock change relating to the distribution package 17e in said storage location 16e). In this embodiment, too, the storage for drug distribution packages may be formed of only one or several cabinets 40 next to and/or on top of each other (without the chest 12 of drawers). Furthermore, one cabinet 40 may comprise one or more rack units 42 closable by a door 19a.

In the embodiment of FIG. 10, the distribution packages 17e are receptacles which are open on top, and dimensioned according to the dimensions of the shelves 43 in the rack unit 42 so that it is not possible to add and remove drugs from the distribution package 17e except by pulling said distribution package 17e at least partly out of the storage location 16e (i.e. over the edge of the shelf 43). Thus, in this embodiment the stop device for preventing the addition or removal of drugs from the storage 10 for drug distribution packages is formed by the shelves 43 on top of each other in the rack unit 42.

In another similar embodiment, the removal and addition of distribution packages 17e may also be prevented by locking so that the distribution packages 17e cannot move from their storage locations 16e unless an authorized user has logged in the stock data system of the storage 10 for drug distribution packages, and entered personal data and other data possibly required. The locking could be provided, for example, by a door 19a that covers the front wall of the rack unit 42 completely and is lockable by e.g. an electric lock, or by a distribution package specific locking. The distribution package specific locking could comprise an electronic locking mechanism for inserting a pin for locking the distribution package 17e in place, for example in a hole at the bottom of the distribution package 17e, or in another stopper placed on a side (e.g. a sleeve, hole, or the like, in a wall).

In the system based on the storage 10 for drug distribution packages of FIG. 10, too, logging in may take place either manually or by means of e.g. an electronic ID card or a remote readable identification.

In another embodiment similar to this embodiment, side walls may also be provided between each storage location 16e. Thus, the detecting sensor for detecting the movement may also be placed on either or both of the side walls. In an embodiment, on the other hand, a detecting sensor on the side wall could be used for detecting the movement of two adjacent distribution packages by means of one detecting sensor.

Also, the principle of operation of the stock data system may be in many respects different from that described above. For example, the functions could include e.g. automatic detection of a distribution package and opening of a drawer or cabinet door when a bar code reader is used to read a bar code in the prescription of a patient, telling the stock data system which drug the person has come to pick up. The stock data system may either be specific to the storage for drug distribution packages, to the pharmacy, or, for example in a hospital, be integrated in a system relating to the patients or their medication, whereby the patients' medication data may be automatically linked to the stock data system of the storage for distribution packages so that when drugs are taken from the storage for drug distribution packages, the data about the drugs to be taken is automatically linked to the system controlling the storage for drug distribution packages. Thus, the system, method, and the storage for drug distribution packages according to the invention are not limited to the above presented example embodiments but they can be implemented in various ways within the scope of the appended claims.

1. A system for storing drugs in distribution packages of a storage for drug distribution packages, the system comprising:
a storage for drug distribution packages, comprising storage locations, in which, the drug distribution packages can be placed;
a data processing device having a memory for storing the number of distribution packages and/or drugs in the storage for drug distribution packages, the expiry date of the drugs, and at least one item of drug information, and the personal data of a user of the storage for drug distribution packages;
a stock data system stored in the memory of the data processing device and configured to control the functions of the storage for drug distribution packages, as well as to record stock data entries in the storage for drug distribution packages, and to administer user rights of the stock data system;
at least one detecting sensor placed in storage locations and coupled to the data processing device and configured to detect the movement of a drug distribution package with respect to the storage location,
wherein
the distribution packages and/or the storage locations are equipped with at least one stop device configured to prevent the addition of drugs into a distribution package and the removal of drugs from a distribution package when the distribution package is placed in an appropriate storage location of the storage for drug distribution packages.

2. The system according to claim 1, wherein the storage location is delimited by adjacent distribution packages and/or at least one wall and the storage location has such inner dimensions, that it only accommodates one distribution package of said storage for drug distribution packages.

3. The system according to claim 1, wherein the stop device is the lid of the distribution package, and the distribution packages have such shapes and are placed in such a way with respect to the lids of the distribution packages of the adjacent storage locations, when placed in the storage locations, that the lids of the adjacent distribution packages prevent each other from being opened.

4. The system according to claim 1, wherein the distribution package comprises a receptacle part and a lid closable by a fastening and/or closing mechanism requiring a rotating motion, and in which system, the unscrewing of the lid of the distribution package is prevented when the distribution package is placed in the storage location.

5. The system according to claim 4, wherein the lids of the distribution packages have a rectangular shape and are placed in such a way with respect to the adjacent distribution packages that when the distribution packages are placed in the storage location, the side edges of the lids are against each other, whereby the unscrewing of the lids is prevented.

6. The system according to claim 1, wherein the distribution package comprises a receptacle part and a lid connected to at least one side of it to be at least partly slidable in one direction on and off this side, along guide bars in the distribution package, and wherein the sliding of the lid of the distribution package is prevented by preventing the sliding of the lid in said direction when the distribution package is placed in the storage location.

7. The system according to claim 1, wherein the storage location comprises one or more walls extending around the distribution package, so that it prevents the removal of drugs from the distribution package and the addition of drugs into the distribution package when the distribution package is placed in an appropriate storage location of the storage for drug distribution packages.

8. The system according to claim 7, wherein the storage location comprises a bottom and at least three walls extending upwards from the bottom of the storage location and joining the adjacent walls in straight angles.

9. A method for storing drugs in distribution packages of a storage for drug distribution packages, wherein the storage for drug distribution packages comprises a data processing device and a stock data system therein, the method comprising:
logging in by a user in the stock data system of the storage for drug distribution packages;
removing at least one distribution package from a storage location of the storage for drug distribution packages;
detecting the removal of the distribution package by at least one detecting sensor placed in the storage location; and recording the entry automatically in the stock data system of the storage for drug distribution packages; and alternatively
removing at least one drug from a drug package and adding it into at least one distribution package removed from the storage for drug distribution packages, and closing the distribution package and/or placing it back in a storage location of the storage for drug distribution packages; or
removing at least one drug from at least one distribution package removed from the storage for drug distribution packages, and closing the distribution package and/or placing it back in a storage location of the storage for drug distribution packages;
wherein
the addition and removal of drugs from the distribution package is prevented by at least one stop device in the storage location and/or the distribution package when the distribution package is placed in an appropriate storage location of the storage for drug distribution packages.

10. The method according to claim 9, wherein the data of the drugs to be added into the distribution packages are read from a bar code or an identification on the drug package into the stock data system.

11. The method according to claim 9, wherein the distribution package is identified by an identifier on at least one wall of the distribution package when the distribution package is placed in the storage location.

12. The method according to claim 9, wherein the location of the distribution package in the storage for drug distribution packages is automatically updated in the stock data system when the distribution package is replaced in the storage location.

13. A storage for drug distribution packages, comprising:
a frame equipped with storage locations, wherein at least one distribution package suitable for storing and distributing of drugs can be placed in at least one storage location;
a data processing device having a memory for storing the number of distribution packages and/or drugs in the storage for drug distribution packages, the expiry date of the drugs, and at least one item of drug information and the personal data of a user of the storage for drug distribution packages;
a stock data system stored in the memory of the data processing device and configured to control the storage for drug distribution packages and to record stock data entries in the storage for drug distribution packages, as well as user identification data;
at least one detecting sensor placed in the storage locations, connected con-Fleeted to the data processing device, and configured to detect a movement of a distribution package with respect to the storage location when the distribution package is placed in the storage location;
wherein
the distribution packages and/or the storage locations are provided with at least one stop device configured to prevent the addition and removal of drugs from the distribution package when the distribution package is placed in an appropriate storage location of the storage for drug distribution packages.

14. The storage for drug distribution packages according to claim 13, wherein the storage locations are placed in drawers to be fitted in a chest of drawers formed in the frame of the storage for drug distribution packages.

15. The storage for drug distribution packages according to claim 14, wherein the storage locations are determined according to the outer dimensions of the distribution packages placed adjacent to and one after each other in the drawer, as well as the inner dimensions of the drawer, so that a space of an appropriate size is provided for one distribution package in each storage location.

16. The storage for drug distribution packages according to claim 15, wherein the lid of the distribution package has a rectangular shape and has such outer dimensions that the lid of the distribution package substantially covers the open top of the storage location.

17. The storage for drug distribution packages according to claim 13, wherein the storage locations are placed on shelves of a closet constituting the whole storage for drug distribution packages, or a cabinet forming part of the same.

18. The storage for drug distribution packages according to claim 17, wherein the storage locations are formed on the shelves in such a way that the distribution packages can be placed in a primarily horizontal position next to and on top of each other on the shelves.

19. The storage for drug distribution packages according to claim 17, wherein the storage locations are formed on the shelves, in such a way, that the distribution packages can be placed in a primarily vertical position next to and on top of each other on the shelves.
